(19) United States
(12) Patent Application Publication
(10) Pub. No.: US 2003/0150793 A1
(43) Pub. Date: Aug. 14, 2003

(54) Filtration unit comprising calendered leukocyte-removing layers

(76) Inventors: Thierry Verpoort, Mouvaux (FR); Stephane Chollet, Mouvaux (FR)

Correspondence Address: BAKER BOTTS, LLP, 910 LOUISIANA, HOUSTON, TX, 77002-4995, US

(21) Appl. No.: 10/364,540

(22) Filed: Feb. 11, 2003

(30) Foreign Application Priority Data
Feb. 13, 2002 (FR) FR02/01776

(51) Int. Cl. B01D 29/00

(52) U.S. Cl. 210/489; 210/483; 210/488

(57) ABSTRACT

The present invention is directed to a filtration unit intended to allow the removal of leukocytes from a fluid such as blood or a blood component. The filtration unit contains a porous element comprising a medium for the removal of leukocytes by adsorption and filtration of the leukocytes. The medium comprises a number of layers of a first type which are formed from at least one porous non-woven material. At least one layer has been pressed by calendering prior to the stacking thereof. The at least one calendered layer is disposed on the downstream side of the stack. The medium also comprises at least one non-calendered layer. The present invention is also directed to a bag-based system comprising the filtration unit. The system is in particular arranged for the sterile and closed-circuit filtration of the fluid.

### CROSS REFERENCE TO RELATED APPLICATION

This application claims priority from French Patent Application No. FR 02 01776 filed Feb. 13, 2002 under 35 U.S.C. § 119(a) and is incorporated herein by reference.

### FIELD OF THE INVENTION

The present invention relates to a filtration unit intended to allow the removal of leukocytes from a fluid, and a bag-based system comprising such a filtration unit.

It applies typically to the filtration of blood or a blood component and to the separation and collection of different constituents of the blood in the bag-based system, in particular in closed circuit.

### BACKGROUND OF THE INVENTION

Filtration units are already known which comprise an outer casing provided with at least one input aperture and at least one output aperture between which the fluid to be filtered flows in one direction, the casing containing a porous element comprising a medium for the removal of leukocytes by adsorption and filtration of the leukocytes.

In such units, illustrated for example by the document EP-A-0 526 678, it is conventional to use, as the leukocyte-removal medium, a stack of filtering layers formed from a porous non-woven material.

This is because, in this type of filtration—referred to as depth filtration—the capacity of the filter medium to retain the leukocytes is a function in particular of the amount of material through which the fluid passes, and therefore of the thickness of the filter medium. In addition, the disposition of a plurality of fine layers makes it possible to improve the leukocyte-removal efficiency compared with a filter medium of the same total thickness formed from a single layer.

In order to improve the effectiveness of this type of filtration, that is to say increase the quantity of leukocytes retained by the leukocyte-removal medium, consideration has therefore been given to increasing the number of stacked layers.

This solution has a number of drawbacks, however.

First, it implies an increase in the overall size of the filter which, generally speaking, is not desirable. In addition, it leads to an increase in the dead volume of the filtration unit, that is to say the amount of fluid remaining in the filtration unit after filtration, this fluid consequently being either lost or difficult to recover. In particular, in filtration units intended to filter a small amount of fluid, this constraint quickly becomes prohibitive.

Next, the increase in the number of layers causes an appreciable decrease in the flow rate of the fluid passing through the leukocyte-removal medium by gravity, and therefore increases the filtration time accordingly.

Furthermore, the applicant discovered that, from a certain value, this increase no longer had a notable positive effect on the quantity of leukocytes retained by the leukocyte-removal medium.

### SUMMARY OF THE INVENTION

The invention therefore aims to remedy these drawbacks by proposing in particular a unit having an improved and adaptable filtration capacity, without adversely affecting the filtration flow rate, the size of the filtration unit and its dead volume. In addition, the filtration unit can be integrated into a bag-based system, in particular in closed circuit, in order to allow, in a simple manner, the separation and collection of different constituents of the blood.

To that end, and according to a first aspect, the invention proposes a filtration unit intended to allow the removal of leukocytes from a fluid such as blood or a blood component, of the type comprising an outer casing provided with at least one input aperture and at least one output aperture between which the fluid to be filtered flows in one direction, the casing containing a porous element comprising a medium for the removal of leukocytes by adsorption and filtration of the leukocytes, said medium comprising a number of layers of one and the same type which are formed from at least one porous non-woven material, in which at least one layer has been pressed by calendering prior to the stacking thereof, said at least one calendered layer being disposed on the downstream side of the stack, while the medium comprises at least one non-calendered layer.

According to a second aspect, the invention proposes a bag-based system for the removal of leukocytes from a fluid such as blood or a blood component, which comprises a bag for collecting the filtrate, said bag being connected, by means of a tube and at an input aperture, to an output aperture of a filtration unit as described above.

### BRIEF DESCRIPTION OF THE DRAWINGS

Other objects and advantages of the invention will emerge during the following description given with reference to the accompanying drawings.

FIG. 1 depicts, in a front view, a filtration unit according to one embodiment of the invention.

FIG. 2 depicts schematically and in section along the line II-II, the filtration unit of FIG. 1.

FIG. 3 depicts, in a schematic front view, a bag-based system for the removal of leukocytes from a fluid such as blood or a blood component, according to a first embodiment.

FIG. 4 depicts a bag-based system according to a variant of the embodiment of FIG. 3.

FIG. 5 depicts, in a schematic front view, a bag-based system for the sterile and closed-circuit removal of leukocytes from a fluid such as blood or a blood component, according to a first embodiment.

FIG. 6 depicts, in a schematic front view, a bag-based system for the sterile and closed-circuit removal of leukocytes from a fluid such as blood or a blood component, according to a second embodiment.

### DETAILED DESCRIPTION OF THE INVENTION

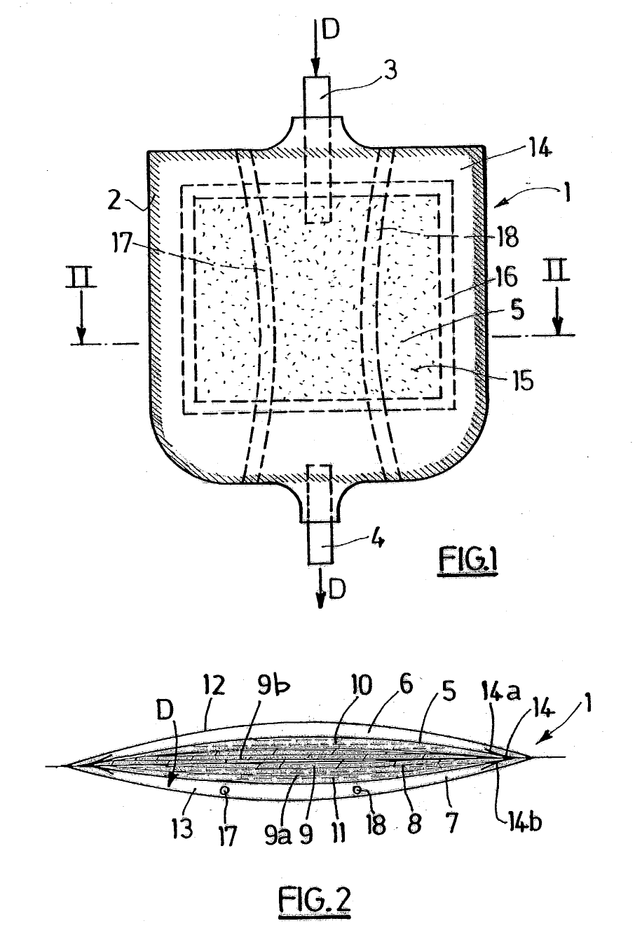

FIGS. 1 and 2 depict a filtration unit 1 intended to allow the removal of leukocytes from a fluid such as blood or a blood component. Blood component means in particular red corpuscles, possibly concentrated and/or in suspension, blood platelets, possibly concentrated and/or in suspension, or blood plasma, possibly poor or rich in platelets.

The blood or a blood component, after its collection and its separation in the case of a component, is in particular intended to be transfused into a patient requiring it.

During this transfusion, it is well known that the leukocytes are undesirable in that they are liable to cause in the patient adverse and/or potentially dangerous reactions.

This is why it is recommended, indeed required in certain countries, that the leukocytes be removed from the blood or blood component prior to the transfusion thereof, at a given efficiency. To date, the optimum solution for eliminating the leukocytes is to filter the blood or blood component through a filtration unit provided with a leukocyte-removal medium.

In the embodiment depicted in FIGS. 1 and 2, the filtration unit 1 comprises an outer casing 2 provided with an input aperture 3 for receiving the fluid to be filtered, and an output aperture 4 for collecting the filtrate, between which the fluid to be filtered flows in a direction D.

The unit 1 also comprises a porous element 5 which is disposed in the outer casing 2 so as to form an input compartment 6 in communication with the input aperture 3 and an output compartment 7 in communication with the output aperture 4.

In the description, the terms “input”, “output”, “upstream” and “downstream” are defined with respect to the direction of movement of the fluid in the filtration unit 1 (see the arrows D shown in FIGS. 1 and 2).

When the filtration unit 1 is supplied with fluid by means of the input aperture 3, said fluid fills the input compartment 6 and then passes through the porous element 5 in order to be collected in the output compartment 7. Next, the filtrate can be collected by means of the output aperture 4.

The porous element 5 comprises a medium 8 for the removal of leukocytes by adsorption and filtration of the leukocytes. The leukocyte-removal medium 8 comprises a number of layers 9 of a first type which are formed from at least one porous non-woven material. Type of layers means layers of material having substantially the same composition, porosity and physico-chemical properties, that is to say substantially the same leukocyte-retention capacity.

According to one embodiment, the layers 9 can be stacked on the downstream side of the leukocyte-removal medium 8 in the direction of flow D of the fluid.

According to the invention, at least one and not all of these layers 9 has been pressed by calendering, in particular cold calendering, prior to the stacking thereof, the calendered layer or layers 9a being disposed on the downstream side of the stack. The stack therefore comprises, from upstream to downstream, at least one noncalendered layer 9b and at least one calendered layer 9a, said layers 9a, 9b all being of the same type.

This particular embodiment makes it possible to obtain a leukocyte-removal medium 8 of which the capacity for adsorption and filtration of the leukocytes is improved compared with a stack of non-calendered layers. This is because the calendering makes it possible in particular to reduce the mean porosity and air permeability of the layer, which increases its leukocyte-retention capacity. The applicant also discovered that, by using a leukocyte-removal medium 8 according to the invention, the time between the fluid being taken and the filtration thereof could be increased without substantially reducing the leukocyte-removal level, for example when this time is 18 hours a satisfactory leukocyte-removal level is still obtained.

Moreover, compared with a stack of layers which have all been calendered, the invention makes it possible to limit the risks of clogging of the leukocyte-removal medium 8 and to maintain a flow rate and therefore an optimal filtration time.

In addition, according to the invention, the number of calendered layers 9a can be adjusted according to the leukocyte-removal efficiency desired or required by the different national legislations.

Finally, the solution proposed by the invention makes it possible to combine the advantages mentioned above with very simple production of the stack since the calendered layers 9a or non-calendered layers 9b are of the same type.

In a variant of the embodiment depicted in FIGS. 1 and 2, the leukocyte-removal medium 9 can also comprise at least one layer of at least a second type, said layer or layers being stacked on the layers 9 of the first type, on the upstream side or the downstream side thereof.

In particular, the layer types can be different by the nature of the material forming them and/or by their physicochemical properties.

According to one embodiment, the mean porosity of the stacked layers decreases continuously or step-wise in the direction of flow. Thus, it is possible to optimize the leukocyte-removal efficiency while reducing the risks of clogging of the leukocyte-removal medium 8.

The porous element 5 can also comprise a pre-filter 10 and/or a post-filter 11, disposed respectively on the upstream side and the downstream side of the leukocyte-removal medium 8. The pre-filter 10 and/or the post-filter 11 can be formed from at least one layer of a non-woven material.

According to a first embodiment, the material or materials forming the layers 9 is/are hydrophilic, in particular made of cellulose or its derivatives, for example cellulose acetate.

According to a second embodiment, the material or materials forming the layers 9 is/are chosen from the group comprising polymers or copolymers based on polypropylene, polyester, polyamide, high or low density polyethylene, polyurethane, polyvinylidene fluoride, polyvinylpyrrolidone and their derivatives.

These polymeric products are not generally naturally hydrophilic and must be treated by physical and/or chemical methods, in order to give them said hydrophilic properties.

These treatments consist for example of grafting hydrophilic substituents, for example hydroxyl or carboxylic type groups, onto the polymer, according to known methods.

Such polymers made hydrophilic by physical and/or chemical treatment are available on the market.

A description is given below, in connection with FIGS. 1 and 2, of one embodiment of a filtration unit 1.

In the embodiment depicted, the outer casing 2 is flexible and formed by the assembly of two sheets 12, 13 of flexible plastic material assembled with one another, for example by welding, on their periphery.

The porous element 5 is held in the outer casing 2 by deformable impervious association means which are formed from a flexible frame 14.

The flexible frame 14 is formed by an assembly of two sheets 14a, 14b, for example plasticized sheets, between which the porous element 5 is placed.

These two sheets 14a, 14b are perforated in their central part and each have at least one opening 15 allowing passage of the fluid to be filtered.

The two sheets 14a, 14b are fixed to one another preferably in the region of the periphery of the porous element 5, for example by a weld seam 16, made through the porous element 5, providing both fixing of the porous element 5 and also sealing.

The welding of the sheets 14a, 14b through the porous element 5 causes a compression, forming an impervious seam around the porous element 5.

The flexible frame 14 is welded on its periphery with the outer sheets 12, 13 forming the outer casing 2, these being welded to one another over their entire circumference and in the region of their periphery, thus providing sealing.

When this welding is performed, the input aperture 3, formed from a portion of tube, is disposed on one side of the flexible frame 14 and the output aperture 4, formed from another portion of tube, is disposed on the other side of the flexible frame 14.

Thus, the input compartment 6 formed between one sheet 12 and the porous element 5 is in communication with the input aperture 3, and the output compartment 7 formed between the other sheet 13 and the porous element 5 is in communication with the output aperture 4.

In order to avoid the porous element 5 sticking against the outer casing 2, and thus interfering with the flow of the fluid, two spacing rods 17, 18 are placed inside the output compartment 7, between the porous element 5 and the outer casing 2.

These two rods 17, 18 keep the output compartment 7 clear of the porous element 5 and thus avoid the porous element 5 being flattened against the inner wall of the outer sheet 13.

The rods 17, 18 can be produced from flexible tubes welded for example at the inner wall of the sheet of the outer casing 2, for example in the region of the peripheral weld.

It is self-evident that the number of spacing rods 17, 18 can vary, depending for example on the dimensions of the filtration unit 1.

For example, provision of a single spacing rod folded so as to form a loop inside the output compartment 7 can be envisioned.

Preferably, flexible rods 17, 18 are used, in order not to interfere with the possibilities of folding the filtration unit 1.

In another embodiment (not depicted), the outer casing 2 is rigid, for example made of a rigid plastic material such as polycarbonate.

Two example embodiments of a porous element 5 for a filtration unit 1 according to the invention are given below.

### EXAMPLE 1

The porous element 5 comprises from upstream to downstream and stacked one upon another:

4 layers of non-woven material made of polyester each having a thickness e of the order of 400 μm, a mean porosity p=35 μm and an air permeability P lying between 1000 and 5000 1/m²/s, as a pre-filter 10;

22 layers 9b of non-woven material made of meltblown polypropylene each having 250 μm<e<400 μm, 8.5 μm<p<10 μm and 130 1/m²/s<P<200 1/m²/s;

2 layers 9a of non-woven material made of meltblown polypropylene of the same type 9 as the preceding 22 layers 9b, which have been calendered separately so as to each have 130 μm<e<250 μm, 7 μm<p<9 μm and 70 1/m²/s<P<130 1/m²/s;

1 layer of non-woven material made of meltblown polyester each having a thickness e of the order of 400 μm, p=35 μm and 1000 1/m²/s<P<5000 1/m²/s, as a post-filter 11.

In one particular example, this porous element 5 has a filtration surface between 50 and 58 cm², for example equal to 55 cm², so as to allow the filtration of 450 ml of fluid with a retention level of 4.8 log (that is to say that the quantity of leukocytes is divided by 10^{4.8} in passing through the porous element 5) compared with 4.3 with a similar porous element in which the two layers 9a have not been calendered, with similar dead volume and filtration time.

Of course, depending on the leukocyte-removal objectives to be achieved, a different number of layers 9 can be calendered.

### EXAMPLE 2

The porous element 5 comprises from upstream to downstream and stacked one upon another:

2 layers of non-woven material made of polyester each having a thickness e of the order of 400 μm, a mean porosity p=35 μm and an air permeability P lying between 1000 and 5000 1/m²/s, as a pre-filter 10;

2 layers of non-woven material made of meltblown polypropylene each having 250 μm<e<400 μm, 10 μm<p<20 μm and 250 1/m²/s<P<400 1/m²/s;

18 layers 9b of non-woven material made of meltblown polypropylene each having 250 μm<e<400 μm, 8.5 μm<p<10 μm and 130 1/m²/s<P<200 1/m²/s;

2 layers 9a of non-woven material made of meltblown polypropylene of the same type 9 as the preceding 18 layers 9b, which have been calendered separately so as

to each have 130 μm<e<250 μm, 7 μm<p<9 μm and 70 1/m²/s<p<130 1/m²/s;

1 layer of non-woven material made of meltblown polyester each having a thickness e of the order of 400 μm, p=35 μm and 1000 1/m²/s<P<5000 1/m²/s, as a post-filter 11.

In one particular example, this porous element 5 has a filtration surface between 15 and 35 cm², for example equal to 20 cm², so as to allow the filtration of 200 ml of fluid.

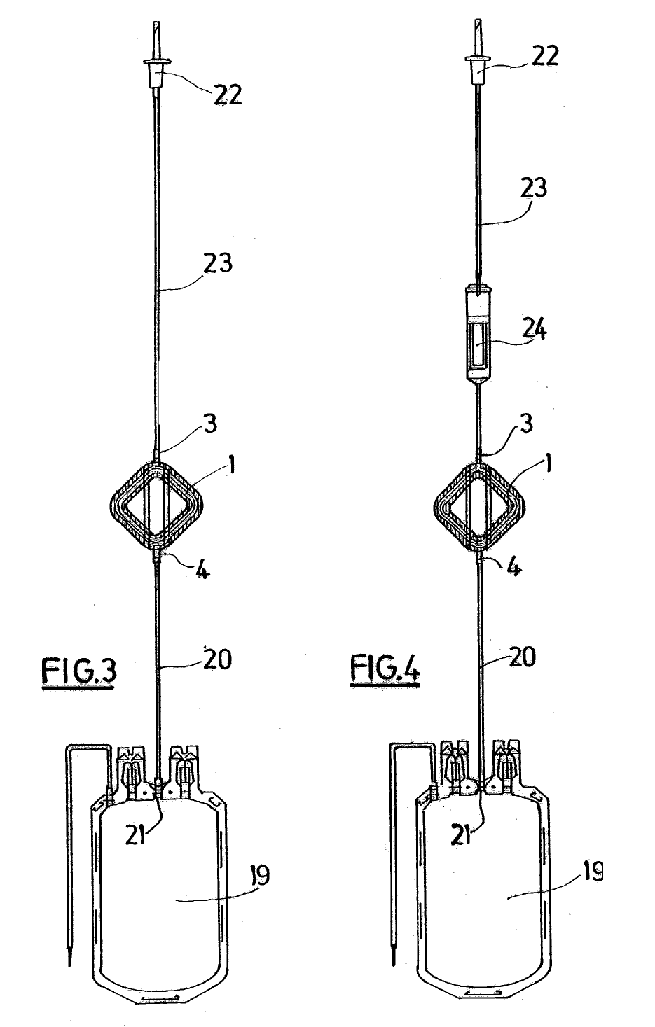

A description will now be given, in connection with FIGS. 3 and 4, of a first embodiment of a bag-based system for the removal of leukocytes from a fluid such as blood or a blood component which comprises a bag 19 for collecting the filtrate, said bag being connected, by means of a tube 20 and at an input aperture 21, to an output aperture 4 of a filtration unit 1 according to the invention.

The system also comprises means 22 of connection with a bag containing the fluid to be filtered which are connected, by means of a tube 23, to an input aperture 3 of the filtration unit 1.

Thus the fluid, once gathered, can be introduced into the bag-based system in order to be filtered by means of the filtration unit 1, the filtrate then being collected in the bag 19.

In the variant depicted in FIG. 4, a microaggregate filter 24 is connected to the system upstream of the filtration unit 1.

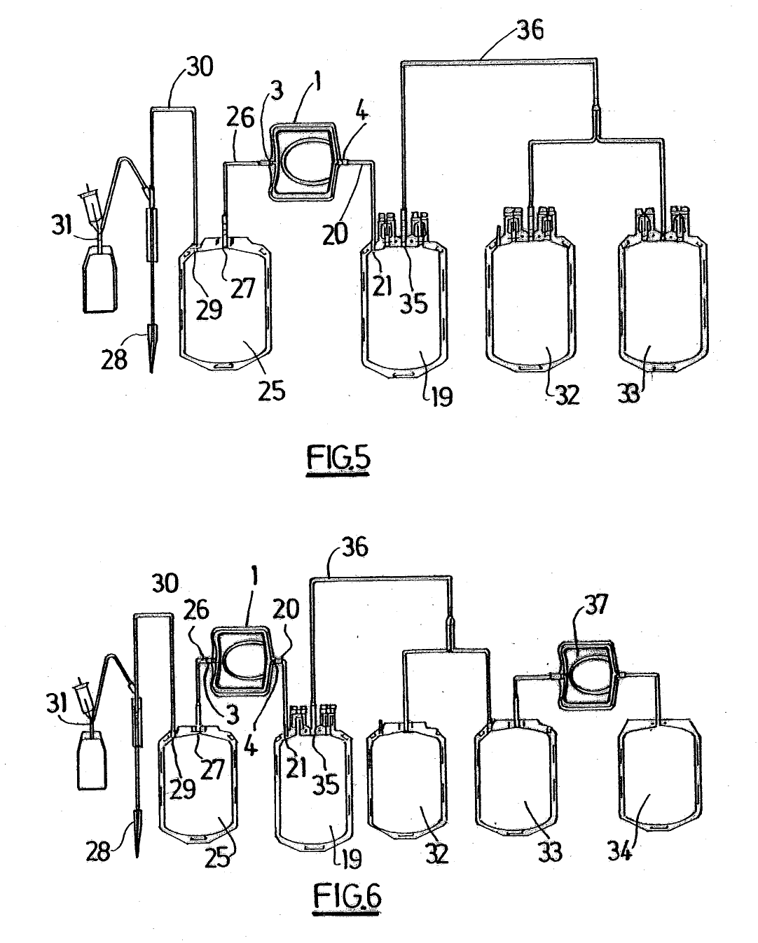

A description is given below, in connection with FIGS. 5 and 6, of a first and a second embodiment of a bag-based system for the sterile and closed-circuit removal of leukocytes from a fluid such as blood or a blood component, said system comprising a filtration unit 1 according to the invention.

To that end, the bag-based systems comprise a gathering bag 25 intended to contain the fluid to be filtered which has previously been filled with a preservation solution for example of CPD type, said bag 25 being connected by means of a tube 26 and at one of its output apertures 27 to the input aperture 3 of the filtration unit 1 and a collecting bag 19 intended to receive the filtrate, said bag 19 being connected by means of a tube 20 and at one of its input apertures 21 to the output aperture 4 of said filtration unit 1.

The bag-based systems in addition comprise means 28 of taking whole blood connected to an input aperture 29 of the bag 25 by means of a tube 30 provided with a device 31 for collecting a sample of blood which has been taken.

The bag-based systems also comprise a set of satellite bags 32-34 connected to an output aperture 35 of the bag 19 by means of a tube 36.

The system according to the first embodiment (FIG. 5) comprises two satellite bags 32, 33, one 32 of which contains a solution for preserving red corpuscles for example of SAGM type. It makes it possible, after sterilization thereof, to successively carry out in closed circuit the following steps:

collection of whole blood in the gathering bag 25;

filtration of the whole blood;

centrifuging of the collecting bag 19;

collection of the different constituents of the blood in the bags 19, 33, namely a concentrate of red corpuscles with the preservation solution added in the bag 19 and plasma in the bag 33.

The system according to the second embodiment (FIG. 6) comprises three satellite bags 32-34, one 32 of which contains a solution for preserving red corpuscles for example of SAGM type and a unit 37 for filtering plasma which is connected between the bags 33, 34. It makes it possible, after sterilization thereof, to successively carry out in closed circuit the following steps:

collection of whole blood in the gathering bag 25;

filtration of the whole blood;

centrifuging of the collecting bag 19;

collection of the different constituents of the blood in the bags 19, 33, namely a concentrate of red corpuscles with the preservation solution added in the bag 19 and plasma in the bag 33;

filtration of the plasma through the filtration unit 37 so as to eliminate the cellular elements;

collection of the filtered plasma in the bag 34.

In a variant, the tubes are flexible, and can be cut and welded in order to make it possible, after the filtration and before the centrifuging, to separate the filtration unit 1 from the bag-based system.

As those of ordinary skill in the art will recognize, the filtering unit 1 can take many forms and may be modified in ways not specifically disclosed herein. It is intended, however, that such forms and modifications be encompassed by the present invention as set forth in the appended claims hereto. Furthermore, those of ordinary skill in the art will recognize that the present invention may be utilized in many other blood collection, separation and storage systems not specifically disclosed herein.

1. A filtration unit for removing leukocytes from a fluid such as blood or a blood component, said filtration unit comprising an outer casing provided with at least one input aperture and at least one output aperture between which the fluid to be filtered flows in one direction, said outer casing comprising a porous element comprising a medium for the removal of leukocytes by adsorption and filtration of the leukocytes, said medium comprising a plurality of stacked layers having an upstream side and a downstream side of a first type which are formed from at least one porous non-woven material, wherein at least one layer has been pressed by calendering prior to the stacking thereof, said at least one calendered layer being disposed on the downstream side of the stack, the medium further comprising at least one non-calendered layer.

2. A filtration unit according to claim 1, wherein the plurality of layers are stacked on the downstream side of the porous element in the direction of flow.

3. A filtration unit according to claim 1, wherein the leukocyte-removal medium also comprises at least one layer of a second type, said layer or layers being stacked on the layers of the first type, on the upstream side or the downstream side thereof.

4. A filtration unit according to claim 3, wherein the first and the second layer types are formed of a different material.

5. A filtration unit according to claim 3, wherein the first and the second layer types have different physico-chemical properties.

6. A filtration unit according to claim 3, wherein the mean porosity of the stacked layers decreases continuously or step-wise in the direction of flow.

7. A filtration unit according to claim 1, wherein the porous element also comprises a pre-filter disposed on the upstream side of the leukocyte-removal medium.

8. A filtration unit according to claim 1, wherein the porous element also comprises a post-filter disposed on the downstream side of the leukocyte-removal medium.

9. A filtration unit according to claim 1, wherein the material or materials forming the layers is/are hydrophilic.

10. A filtration unit according to claim 1, wherein the material or materials forming the layers is/are chosen from the group consisting of polymers or copolymers based on polypropylene, polyester, polyamide, high or low density polyethylene, polyurethane, polyvinylidene fluoride, polyvinylpyrrolidone and their derivatives, said material or materials being made hydrophilic by physical or chemical treatment.

11. A filtration unit according to claim 1, wherein the outer casing is formed from two sheets of flexible plastic material assembled on their periphery.

12. A filtration unit according to claim 1, wherein the porous element is held in the outer casing by deformable impervious association means.

13. A filtration unit according to claim 12 wherein the association means comprise a flexible frame in which the porous element is held, the flexible frame being formed from two perforated flexible sheets between which the porous element is placed, said sheets being fixed in the region of the periphery of the porous element by a weld seam made through the porous element and fixed in the region of the periphery of the outer casing by welding with the sheets forming the outer casing.

14. A bag-based system for the removal of leukocytes from a fluid such as blood or a blood component, comprising a bag for collecting filtrate, said bag being connected, by a tube and at an input aperture to an output aperture of a filtration unit, said filtration unit comprising an outer casing comprising a porous element comprising a medium for the removal of leukocytes by adsorption and filtration of the leukocytes, said medium comprising a plurality of stacked layers, having an upstream side and a downstream side, which are formed from at least one porous nonwoven material, wherein at least one layer has been pressed by calendering prior to stacking thereof, said at least one calendered layer being disposed on the downstream side of the stack, the medium further comprising at least one non-calendered layer.

15. A bag-based system according to claim 14, further comprising a gathering bag intended to contain the fluid to be filtered, said gathering bag being connected, by a tube and at an output aperture to an input aperture of said filtration unit, so as to allow sterile and closed-circuit filtration of the fluid.

16. A bag-based system according to claim 14, further comprising a set of satellite bags connected by a tube to an output aperture of the collecting bag.

17. A bag-based system according to claim 16, wherein the set of satellite bags comprises at least two bags and another filtration unit, said another unit being adapted for fluidic communication with the two bags of the set.

18. A bag-based system according to claim 15, further comprising a means of taking fluid connected to an input aperture of the gathering bag.

19. An apparatus for removing leukocytes from blood or a blood component, comprising a porous element comprising, a leukocyte removing medium formed of a plurality of stacked layers, at least one of said plurality of stacked layers being calendered.

20. An apparatus, according to claim 19, further comprising a pre-filter disposed on an upstream side of the medium and a post-filter disposed on a downstream side of the medium.
